(19) United States
(12) Patent Application Publication
(10) Pub. No.: US 2022/0018771 A1
(43) Pub. Date: Jan. 20, 2022

(54) SYSTEM AND METHOD FOR DETECTING A GIVEN GAS SPECIES PRESENT IN A GASEOUS SAMPLE USING GAS FILTER CORRELATION SPECTROSCOPY

(71) Applicant: INSTITUT NATIONAL D'OPTIQUE, Québec (CA)

(72) Inventors: François BABIN, Quebec (CA); Jean-François GRAVEL, Jonquière (CA); Pascal DUFOUR, Quebec (CA)

(21) Appl. No.: 16/931,474

(22) Filed: Jul. 17, 2020

(51) Int. Cl. G01N 21/64; G01N 21/65; G01N 33/00

(52) U.S. Cl. CPC G01N 21/64; G01N 2201/0668; G01N 33/0036; G01N 21/65

(57) ABSTRACT

There is described a method for detecting a given gas species present in a gaseous sample. The method generally has splitting a primary optical pulse into first and second optical pulses, the primary optical pulse having a duration and carrying optical power within an excitation spectrum encompassing at least one absorption spectral band of the given gas species, the first optical pulse being propagated across an optical gas filter unit containing an amount of the given gas species and attenuating the first optical pulse at the at least one absorption band, one of i) the primary optical pulse and ii) the first and second optical pulses being propagated across the gaseous sample, and temporally delaying the first and second optical pulses from one another; measuring signal values of the delayed optical pulses; and detecting the presence of the given gas species in the gaseous sample based on the signal values.

### FIELD

The improvements generally relate to the optical detection of a given gas species in a gaseous sample and more specifically relate to optical detection involving gas filter correlation spectroscopy.

### BACKGROUND

A gas species is typically characterized by one or more specific spectral absorption bands which indicate at which optical wavelengths or frequencies optical radiation is absorbed by the gas species.

Gas correlation spectroscopy takes advantage of these spectral absorption bands. For instance, one existing gas correlation spectroscopy instrument has an optical source for generating light, an optical detector for detecting the light and generating an output signal, and an optical path extending between the optical source and the optical detector. An optical gas correlation filter unit containing the gas species to be detected is also provided. The optical gas correlation filter unit can be movable between a first position away from the optical path and a second position in the optical path.

In this way, when light is propagated along the optical path and the optical gas correlation filter unit is moved at the first position, the optical detector generates a first signal having a first amplitude (A₁). However, when the optical gas correlation filter unit is moved at the second position, the gas species contained inside the optical gas correlation filter unit absorbs light at the spectral absorption bands of the gas species, which leads the light detector to generate a second signal having a second amplitude (A₂), generally smaller than the first signal amplitude due to absorption in the gas filter unit.

As can be understood, one can determine whether the gas species is present along the optical path by monitoring a difference between the first and second signal amplitudes as the optical gas correlation filter unit is moved between the first and second positions. Indeed, when a gaseous sample containing the gas species is disposed along the optical path, the first signal amplitude would decrease, due to such absorption, thus reducing the difference between the first and second signal amplitudes.

Recently, gas correlation spectroscopy instruments with no moving parts have been developed for increased robustness. Typically, these gas correlation spectroscopy instruments have a first optical path having an optical gas correlation filter unit and a second optical path with no optical gas filter unit. These gas correlation spectroscopy instruments generally rely on either two distinct optical sources or two distinct detectors, or both, one for each of the two paths. One aspect that inconveniently affects the design of these gas correlation spectroscopy instruments is the stability of the instrument offset over time. Since there can be two optical paths and two optical signals, computations and/or the electrical fluctuations generate an offset value between the first and second signal amplitudes when the gaseous sample does not contain the gas species to be detected. In many gas correlation spectroscopy instruments, the offset is nulled (i.e., moved to zero) by inserting an attenuator in one of the optical paths.

However, using two distinct optical sources tends to create a varying offset because of dissimilar behaviors of each one of the optical sources with ageing, temperature fluctuations and so forth. Moreover, using two distinct detectors tends to create a varying offset because of differences in electronic offsets, in spectral responses which vary over time differently, in temperature responses, in gain fluctuations, in linearity variations and so forth.

Although existing gas correlation spectroscopy instruments are satisfactory to a certain degree, there remains room for improvement, especially in alleviating the above-mentioned drawbacks.

### SUMMARY

In an aspect, there are described methods and systems for detecting a given gas species present in a gaseous sample. More specifically, a primary optical pulse is generated and propagated along a primary optical path. The primary optical pulse has a temporal pulse shape, and carries optical power within an excitation spectrum encompassing an absorption spectral band of the given gas species. The primary optical pulse so-propagated is split into a first optical pulse and a second optical pulse such that the first and second optical pulses both have the temporal pulse shape of the primary optical pulse. The first optical pulse is propagated into an optical gas filter unit containing an amount of the gas species to be detected and which attenuates the first optical pulse at the absorption spectral band. Using a time delay unit, the first and second optical pulses are temporally delayed from one another by a time delay which can exceed the duration of the pulses. Along the system, either the primary optical pulse or the first and second optical pulses are propagated across the gaseous sample. Once temporally delayed, signal values indicative of the energy of the first and second optical pulses are detected using at least one optical detector. As can be understood, the presence of the gas species in the gaseous sample can be detected based on the measured signal values.

In accordance with an aspect, there is provided a method for detecting a given gas species present in a gaseous sample, the method comprising: splitting a primary optical pulse into a first optical pulse and a second optical pulse, the primary optical pulse having a duration and carrying optical power within an excitation spectrum encompassing at least one absorption spectral band of the given gas species, the first optical pulse being propagated across an optical gas filter unit containing an amount of the given gas species to be detected and attenuating the first optical pulse at the at least one absorption band, one of i) the primary optical pulse and ii) the first and second optical pulses being propagated across the gaseous sample, and temporally delaying the first and second optical pulses from one another; measuring measurement signal values of the temporally delayed first and second optical pulses using a measurement optical detector; and detecting the presence of the given gas species in the gaseous sample based on the measurement signal values.

In accordance with another aspect, there is provided a system for detecting a given gas species present in a gaseous sample, the system comprising: an optical pulse generator configured to generate a primary optical pulse directed along a primary optical path, the primary optical pulse having a duration and carrying optical power within an excitation spectrum encompassing at least one absorption spectral band of the given gas species; an optical beam splitter configured for splitting the primary optical pulse into a first optical pulse directed along a first arm path and a second optical pulse directed along a second arm path, the first arm path having an optical gas filter unit containing an amount of the given gas species to be detected and configured for attenuating the first optical pulse at the at least one absorption band, one of the first and second arm paths having a time delay unit being configured to cause a time delay between the first and second optical pulses, one of i) the primary optical path and ii) the first and second arm paths extending across the gaseous sample; a measurement optical detector configured for measuring measurement signal values of the first and second optical pulses propagating along the first and second arm paths; and a computer detecting the given gas species in the gaseous sample based on the measurement signal values.

In some specific embodiments, the first and second optical pulses are combined to one another along a common optical path leading to a single optical detector, which can generate signals indicative of the energy of the temporally delayed first and second optical pulses. The methods and systems described herein can thus be convenient as only one optical detector may be used in some embodiments, which can reduce drawbacks associated with monitoring additional components as the responses of these additional components typically drift independently from one another over time. As can be understood, the reduced number of components can also be convenient as the resulting system has a reduced footprint, a reduced weight, is less expensive and requires less power.

In the present description, the terms “light” and “optical” are used to refer to electromagnetic radiation having a wavelength or a wavelength band lying in some specific regions of the electromagnetic spectrum. More particularly, the terms “light” and “optical” are not limited to visible light, but can also include, for example, terahertz, infrared, and ultraviolet wavelength ranges. For example, the terms “light” and “optical” can encompass electromagnetic radiation having a wavelength ranging from a few hundreds of nanometers (nm) to a few tens of micrometers (μm) depending on the application.

In this disclosure, the expressions “optical path”, “primary optical path” or “arm paths” are not to be interpreted in a limiting manner. Rather, the expressions “optical path”, “primary optical path” and “arm paths” are used broadly so as to refer to paths along which an optical pulse (e.g., the primary optical pulse, the first optical pulse, the second optical pulse and so forth) may propagate, in free space and/or in one or more lengths of optical fiber. As will be described with reference to the numerous embodiments illustrated herein, the primary optical path can be split into first and second arm paths using a beam splitter. The arm paths can be further combined to one another back along the primary optical path using an optical beam combiner. As such, the primary optical path can encompass any optical path(s) which is(are) either before or after the first and second arm paths.

In this specification, the expression “gaseous sample” is used to refer to the portion of the gas species being characterized by the method or system independently of whether the gaseous sample is present along an open path or enclosed in a gas cell.

It will be understood that the expression “computer” as used herein is not to be interpreted in a limiting manner. It is rather used in a broad sense to generally refer to the combination of some form of one or more processing units and some form of memory system accessible by the processing unit(s). For instance, the computer can include one or more synchronization units for synchronizing the detection of one or more optical detectors to the generation of optical pulses by one or more optical pulse generators.

It will be understood that the various functions of a computer can be performed by hardware or by a combination of both hardware and software. For example, hardware can include logic gates included as part of a silicon chip of the processor. Software can be in the form of data such as computer-readable instructions stored in the memory system. With respect to a computer, a processing unit, or a processor chip, the expression “configured to” relates to the presence of hardware or a combination of hardware and software which is operable to perform the associated functions.

Many further features and combinations thereof concerning the present improvements will appear to those skilled in the art following a reading of the instant disclosure.

### DESCRIPTION OF THE FIGURES

In the figures,

FIG. 1 is a schematic view of an example of a system for detecting a given gas species present in a gaseous sample, the gaseous sample being housed inside a chamber downstream from an optical assembly comprising a first arm path having an optical gas filter unit and a second arm path having a time delay unit;

FIG. 1A is a schematic view of exemplary emission and collection assemblies used when the gaseous sample is distributed along an open path, in accordance with an embodiment;

FIG. 2 is a graph illustrating the excitation spectrum of a primary optical pulse generated by the system of FIG. 1 (solid line) and the corresponding spectrum of the primary optical pulse after passage through a cell containing an amount of a given gas species (dashed line), in accordance with an embodiment;

FIG. 3A is a graph showing signal as function of time when the gaseous sample does not contain the given gas species, in accordance with an embodiment;

FIG. 3B is a graph showing signal as function of time when the gaseous sample does contain the given gas species, in accordance with an embodiment;

FIG. 4 is a schematic view of an example of a system for detecting a given gas species present in a gaseous sample, shown with a gaseous sample located upstream from an optical assembly comprising first and second arm paths;

FIG. 5 is a schematic view of an example of a system for detecting a given gas species present in a gaseous sample, shown with an optical assembly comprising a first arm path having an optical gas filter unit and a time delay unit;

FIG. 6 is a schematic view of an example of a system for detecting a given gas species present in a gaseous sample, shown with filter elements for fluorescence and/or Raman scattering;

FIG. 7 is a schematic view of another example of a system for detecting a given gas species present in a gaseous sample, shown with a reference optical detector located downstream from an optical power combiner and upstream from the gaseous sample;

FIG. 8 is a schematic view of another example of a system for detecting a given gas species present in a gaseous sample, shown with a first optical assembly upstream from a second optical assembly, both optical assemblies having a respective time delay unit provided in the form of a length of optical fiber;

FIG. 9 is a graph showing signal as function of time as four optical pulses reach an optical detector of the system of FIG. 8;

FIG. 10 is a schematic view of another example of a system for detecting a given gas species present in a gaseous sample, shown with a first optical assembly upstream from a second optical assembly, both optical assemblies having a respective time delay unit provided in the form of a multi-reflection cell;

FIG. 11A is a schematic view of another example of a system for detecting first and second gas species present in a gaseous sample, shown with two optical gas filter units each containing one of the first and second gas species;

FIG. 11B is a schematic view of another example of a system for detecting first and second gas species present in a gaseous sample, shown with a single optical gas filter unit containing the first and second gas species;

FIG. 12 is a schematic view of another example of a system for detecting a given gas species present in a gaseous sample, shown with a first optical assembly upstream from a second optical assembly containing a calibration artefact and filter elements for fluorescence and/or Raman scattering; and

FIGS. 13-15 are schematic views of examples of systems for detecting a given gas species present in the open air.

### DETAILED DESCRIPTION

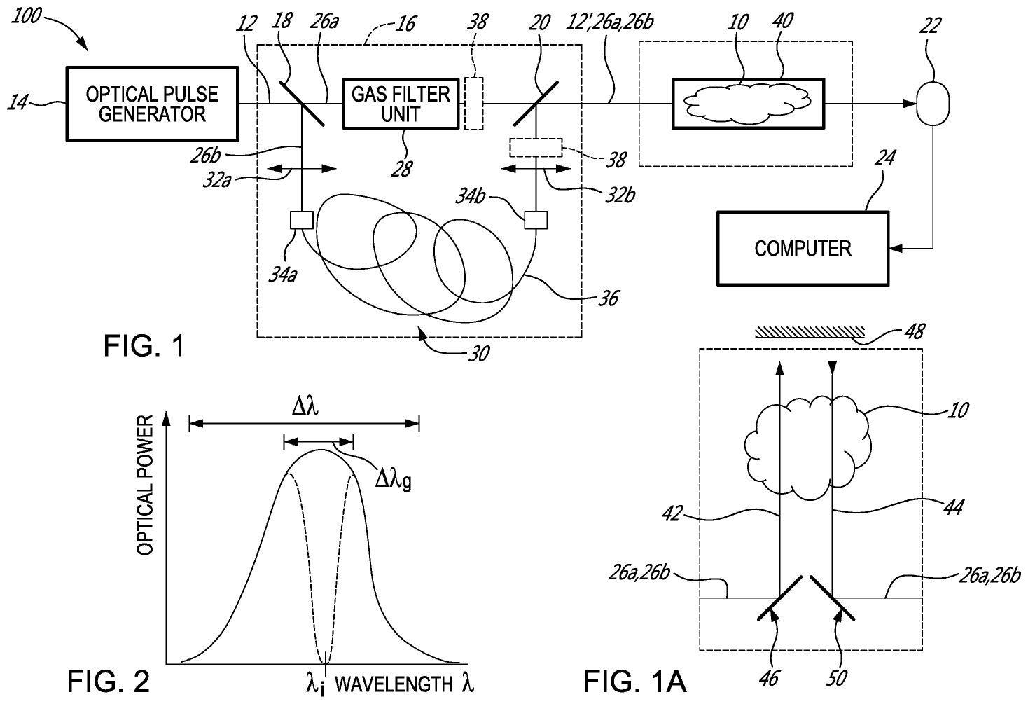

FIG. 1 shows an example of a system 100 for detecting a given gas species present in a gaseous sample 10. The given gas species can be in the form of atoms and/or molecules.

Examples of gas species that can be detected using the system 100 include methane, ethane, alkane(s), benzene, toluene, ethylbenzene, xylene, naphthalene, styrene and/or any gas species that one would want to detect.

Broadly described, the system 100 has an optical pulse generator 14, an optical assembly 16 comprising an optical beam splitter 18 and an optical beam combiner 20, an optical detector 22 and a computer 24.

As shown, the optical pulse generator 14 is configured to generate a primary optical pulse which is propagated along a primary optical path 12. In this example, the primary optical pulse has a temporal pulse shape and a duration Δt, and carries optical power within an excitation spectrum. More specifically, in this example, the excitation spectrum P(λ) is centered on the wavelength A such as shown by the curve plotted in solid line in FIG. 2.

The optical pulse generator 14 is generally chosen so that the excitation spectrum P(λ) of the primary optical pulse is broader than and encompasses an absorption spectrum B(λ) of at least one spectral absorption band of the gas species to be detected. Accordingly, the optical pulse generator 14 can be said to be a broadband optical pulse generator. It is noted that the spectral absorption band can be a narrow spectral band (also referred to as a spectral absorption line) in some embodiments or a collection of adjacent narrow spectral absorption lines, which may be distinguishable from one another, in some other embodiments.

Examples of the optical pulse generator 14 include a pulsed light emitting diode (LED), a broadband pulsed light amplification source such as a pulsed laser diode amplifier, a pulsed doped fiber amplifier source, a pulsed doped crystal or glass amplifier source, a gas discharge lamp and/or any other suitable optical pulse generator that can efficiently generate optical pulses having a satisfactorily short duration, preferably less than a few tens of nanoseconds.

As depicted, the optical beam splitter 18 is disposed along the primary optical path 12 and is configured for splitting the primary optical path 12 into a first arm path 26a and a second arm path 26b. It is intended that when the primary optical pulse is propagated along the primary optical path 12, the optical beam splitter 18 splits the primary optical pulse into a first optical pulse directed along the first arm path 26a and a second optical pulse directed along the second arm path 26b.

As can be understood, the optical beam splitter 18 is such that the first and second optical pulses have the same temporal pulse shape as that of the primary optical pulse. However, due to the splitting, the first and second optical pulses have a lower amount of optical power within the excitation spectrum than that of the primary optical pulse. Accordingly, the splitting is a power splitting of the primary optical pulse.

As shown, the first arm path 26a has an optical gas filter unit 28 containing an amount of the given gas species to be detected and attenuating the first optical pulse at the absorption spectral band of the given gas species. As discussed above, the spectrum B(λ) of the spectral absorption band of the given gas species coincides with at least some of the spectrum P(λ) of the primary optical pulse emitted from the pulse generator 14. The curve plotted in solid line in FIG. 2 is a schematic example of the excitation spectrum P(λ) of the primary optical pulse while the curve plotted in dashed line shows the corresponding spectrum of the primary optical pulse after its passage through the optical gas filter unit 28. The given gas species contained in the optical gas filter unit 28 has a concentration such that when an optical pulse passes through the unit, its optical power within the spectral absorption band is significantly reduced.

Accordingly, as the first optical pulse is propagated across the optical gas filter unit 28, the given gas species of the optical gas filter unit 28 absorbs optical power within the spectral absorption band of the given gas species which results in the first optical pulse having a lesser amount of optical power within the spectrum B(λ) of the spectral absorption band.

In this example, the second arm path 26b has a time delay unit 30. The time delay unit 30 is configured to cause a time delay Δτ between the first optical pulse directed along the first arm path 26a and the second optical pulse directed along the second arm path 26b. More specifically, in this example, the time delay caused by the time delay unit 30 exceeds the duration Δt of the primary optical pulse, i.e. Δτ>Δt. However, in some other embodiments, the time delay may not necessarily exceed the duration Δt. For instance, the time delay may be shorter than the duration Δt but sufficiently long so that the pulses be distinguishable from one another by the optical detector 22.

More specifically, in this example, the second arm path 26b includes a first light converging element 32a configured to couple the second optical pulse into a first end 34a of a length of optical fiber 36, and a second light collimating element 32b configured to receive the second optical pulse from a second end 34b of the length of optical fiber 36 and to propagate the second optical pulse along the second arm path 26b. The light converging and collimating elements can be provided in the form of one or more lenses, objectives, fiber-optic components and the like, depending on the embodiment.

As can be understood, in this example, the time delay caused by the time delay unit 30 is defined by the length of the optical fiber 36. As shown, the length of optical fiber 36 can be curled or wound to occupy a relatively small volume. Moreover, using the length of optical fiber 36 can alleviate the effects of changing air content between the first and second arm paths 26a and 26b. It is noted that the optical transmission spectrum of the length of optical fiber 36 should preferably be relatively flat over the excitation spectrum P(λ) and substantially stable over time.

As shown, the system 100 can have one or more optical attenuators 38 in either one or both of the first and second arm paths 26a and 26b to attenuate either one or both of the first and second optical pulses. The optical attenuators 38 can be variable attenuators in some embodiments or fixed attenuators in some other embodiments.

As illustrated, the optical beam combiner 20 is downstream from the optical gas filter unit 28 and the time delay unit 30, at some position along the first and second arm paths 26a and 26b. The optical beam combiner 20 is configured to combine the first and second arm paths 26a and 26b along a common path 12′ along which the first and second optical pulses propagate.

As can be understood, as the time delay Δτ caused by the time delay unit 30 exceeds the duration Δt, the first and second optical pulses propagating along the first and second arm paths 26a and 26b do not temporally overlap with one another.

In this embodiment, the optical beam splitter 18 is provided in the form of a 50/50 optical beam splitter. Similarly, in this embodiment, the optical beam combiner 20 is provided in the form of a 50/50 optical beam combiner. However, any other suitable optical beam splitter/combiner such as a 80/20 optical beam splitter/combiner, a metallic film beam splitter/combiner, a polarizing beam splitter/combiner, a Polka-Dot beam splitter/combiner and the like can be used. Moreover, in embodiments where the primary optical pulse, the first optical pulse and/or the second optical pulse are propagated along a length of optical fiber, the optical beam splitter 18 and the optical beam combiner 20 can be provided in the form of a fiber coupler.

As depicted, the optical detector 22 is downstream from the optical beam combiner 20 and from the gaseous sample 10, along the combined first and second arm paths 26a and 26b. As such, the optical detector 22 is configured for sequentially detecting the first and temporally delayed second optical pulses. In this way, any variation in the primary optical pulse will be present in both the first and second optical pulses, thus reducing measurement noise when detecting the given gas species or when determining the concentration (e.g., a concentration-length value) of the given gas species in the gaseous sample 10.

In this embodiment, the optical detector 22 is provided in the form of a single photodiode. However, in other embodiments, the optical detector 22 can be a single avalanche photodiode (APD), a single photomultiplier (PM) tube and the like. In alternate embodiments, the optical detector 22 can be an array of detectors, such as an interline CCD camera or a double gate CMOS camera, an example of which will be described below.

As will be understood, the output from the optical detector 22 and the associated electronics can be a signal waveform with respect to time in some embodiments or it can be a signal integrated over a given duration (a gate) in some other embodiments. In all cases, these output signals can be processed, either by electronics connected to the optical detector 22 or by the computer 24, to provide corresponding signal values. Either way, the signal values are said to be measured by the optical detector 22 in this disclosure for ease of reading. It is noted that the signal value of a given signal can correspond to a peak value of the measured signal or to an area under the waveform of the detected signal, which corresponds to the signal generated by the first or second optical pulses plus an offset value generated by the optical detector 22, such as signals generated by unwanted ambient light, electronic offsets or environmental electronic noise. In this embodiment, three signal values are measured: an offset value O which is indicative of the offset measured by the optical detector 22 in absence of any optical pulse; a first signal value V₁ generated by the first optical pulse plus the offset of the optical detector 22, and a second signal value V₂ generated by the second optical pulse plus the offset of the optical detector 22. In this embodiment, the data product of interest can be (V₁−V₂)/(V₁−O) with a properly nulled system and using a single pair of detected pulses.

The computer 24 is configured to detect the given gas species in the gaseous sample based on the measured signal values. In some embodiments, the presence of the given gas species can be detected based on a difference between the measured signal value of the first optical pulse and the measured signal value of the second optical pulse, or on a change of the difference over time. In some embodiments, a concentration of the given gas species in the gaseous sample can be determined based on the relative difference between the first and second signal values V₁ and V₂ and on the offset value O of the optical detector 22. In some embodiments, the difference can be proportional to a subtraction between the first and second signal values V₁ and V₂. However, in some other embodiments, the difference may not be limited to a subtraction, as any other arithmetic relations can be applied to the first and second signal values V₁ and V₂ and still be able to detect the presence of the given gas species.

It is envisaged that the optical beam combiner 20 can be omitted in some other embodiments, in which case the first and second arm paths 26a and 26b are not combined to one another. Although the first and second arm paths 26a and 26b may not be perfectly combined to one another, they can be brought closed and parallel to one another, so as to allow detection by a single optical detector in some alternate embodiments.

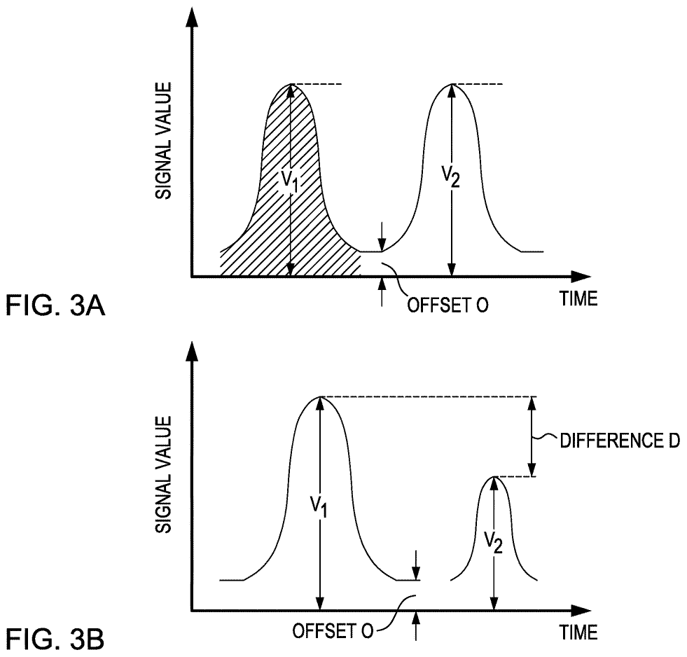

FIGS. 3A and 3B are graphs showing signal as function of time as the first and second optical pulses reach the optical detector 22.

More specifically, FIG. 3A shows signal as function of time as the first and second optical pulses reach the optical detector 22 when the gaseous sample 10 does not contain the given gas species. In this example, the optical attenuator(s) 38 have been configured to attenuate the first optical pulse and/or the second optical pulse so that a difference D between the signal value of the first optical pulse and the signal value of the second optical pulse is null, i.e. the signal values are equal to one another. This process can be referred to as “nulling” the system 100.

Accordingly, when the system 100 is so-nulled, the presence of the given gas species can be detected as the signal value of the second optical pulse will tend to decrease as function of the concentration of the given gas species in the gaseous sample 10, thus increasing the difference between the signal values of the first and second optical pulses. It is understood that the system 100 will not remain nulled forever. However, as the system 100 has a minimal number of components (e.g., one optical pulse generator, one optical detector), there is a lesser number of components which can cause the offset value of the system 100 to drift.

For instance, FIG. 3B shows signal as function of time as the first and second optical pulses reach the optical detector 22 when the gaseous sample 10 does contain the given gas species. As shown, the signal value of the second optical pulse is lower than that of the first optical pulse as some of its optical power has been absorbed by the given gas species of the gaseous sample 10. Accordingly, a change in the difference between the signal value of the first optical pulse and the signal value of the second optical pulse can thus be indicative of the presence of the given gas species in the gaseous sample 10.

Moreover, with proper calibration, the computer 24 can determine a concentration-length value of the given gas species in the gaseous sample based on a change in the difference between the signal values of the first and second optical pulses and on calibration data. A concentration value can be determined based on the concentration-length value and on a length of the gaseous sample 10. For instance, in the case of an open path, the length of the gaseous sample 10 can correspond to the distance between the system 100 and a distant target. In some embodiments, the signal value represents the peak intensity of the pulse received. In some other embodiments, the signal value can represent an integration of the pulse over a given duration, thus giving a value indicative of the pulse energy.

In the embodiment illustrated in FIG. 1, the gaseous sample 10 is enclosed in a transparent chamber 40 which is disposed along the first and second arm paths 26a and 26b. In this case, the first and second arm paths 26a and 26b go through the transparent chamber 40.

However, in some other embodiments such as the one shown in FIG. 1A, the first and second arm paths 26a and 26b include an outgoing path 42 along which the first and second optical pulses travel towards a distant target 48, and a return path 44 along which the first and second optical pulses travel from the distant target 48. As can be understood, the outgoing path 42 and the return path 44 can be referred to as free space paths and/or open paths, as the gaseous sample 10 is not limited by any chamber. In this embodiment, the system 100 includes an emission assembly 46 configured to route the first and second optical pulses travelling along the first and second arm paths 26a and 26b towards the outgoing path 42. The system 100 also includes a collection assembly 50 configured to receive the returning first and second optical pulses travelling along the return path 44 and to route the first and second collected optical pulses back towards the optical detector 22.

In this case, the gaseous sample 10 is distributed along both of the outgoing path 42 and the return path 44 so as to allow detection of the given gas species present in the remote gaseous sample 10. These embodiments are typically referred to as “open path” as the gaseous sample 10 is distributed in the open air through which the first and second optical pulses travel.

In these embodiments, the optical pulse generator 14 and the optical detector 22 are synchronized with one another, allowing the system to pinpoint the first and second pulses in the time and the computer 24 to determine a concentration of the given gas species in the gaseous sample 10. In some embodiments, the system 100 is said to be configured in an open path, an integrated open path or a path integrated light detection and ranging (LIDAR) configuration. These are path integrated configurations which can require a distant target 48 such as a manmade target or a topographical target.

Referring back to FIG. 1, the gaseous sample 10 is disposed along the first and second arm paths 26a and 26b, downstream from the optical beam combiner 20. In this way, the first and second optical pulses propagate across the gaseous sample 10 subsequent to the combining performed by the optical beam combiner 20.

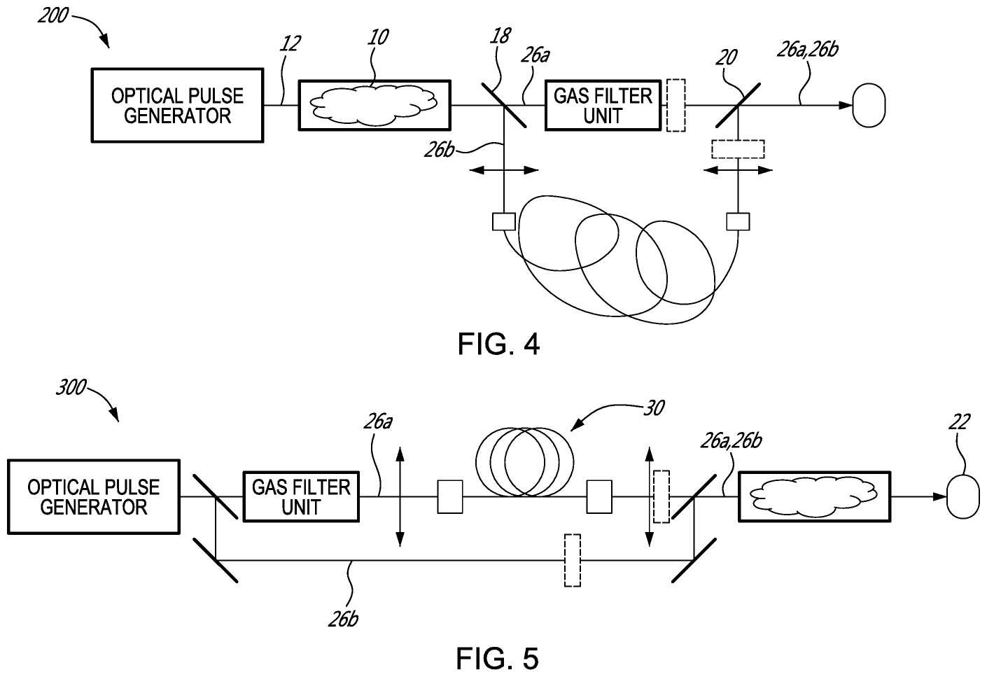

However, the gaseous sample 10 can as well be disposed along the primary optical path 12 upstream from the first and second arm paths 26a and 26b, such as in the system 200 shown in FIG. 4. In this embodiment, the primary optical pulse propagates across the gaseous sample 10 prior to the splitting performed by the optical beam splitter 18.

Referring back to FIG. 1, the time delay unit 30 is disposed along the second arm path 26b, in which case the first optical pulse reaches the optical detector 22 prior to the second optical pulse.

However, the time delay unit 30 can as well be disposed along the first arm path 26a, such as in the system 300 shown in FIG. 5. In this latter embodiment, the second optical pulse reaches the optical detector 22 prior to the first optical pulse.

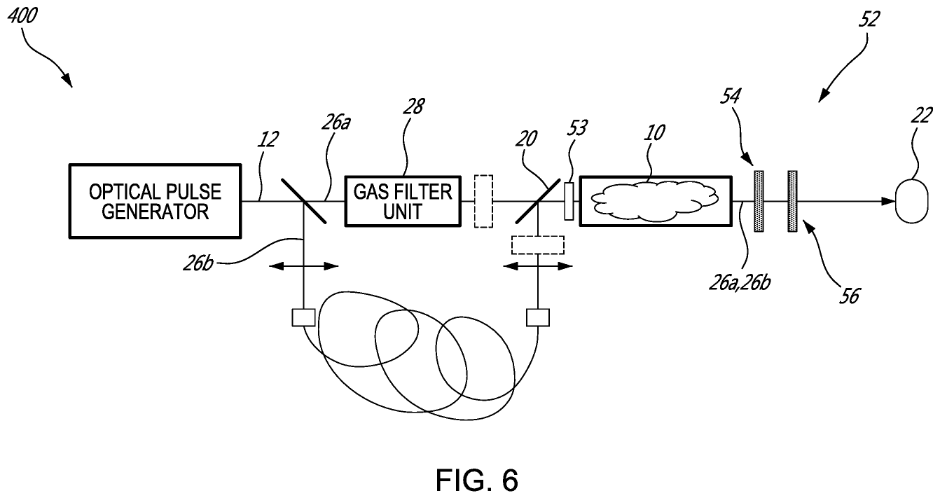

FIG. 6 shows another example of a system 400 for detecting a given gas species present in a gaseous sample 10. As depicted in this embodiment, the gaseous sample 10 is disposed along the first and second arm paths 26a and 26b, downstream from the optical beam combiner 20.

The system 400 of FIG. 6 is similar to the system 100 of FIG. 1. However, in this example, one or more filter elements 52 are located downstream from the gaseous sample 10.

The filter elements 52 are configured to filter out optical power of the excitation spectrum of the first and second optical pulses. In this way, the optical detector 22 can measure signal values which are indicative of fluorescence and/or Raman scattering produced by the first optical pulse and/or produced by the second optical pulse propagating across the gaseous sample 10.

In this example, the system 400 has a bandpass filter 53 for the spectrum of the first and second pulses, which is downstream from the optical beam combiner 20 and upstream from the gaseous sample 10. In this way, only light associated to the first and second optical pulses will reach the gaseous sample 10. Alternatively, the optical detector 22 could have been positioned at an angle relative to a propagation direction of the first and second optical pulses, to allow the optical detector 22 to detect fluorescence and/or Raman scattering as the first and second optical pulses interact with the given gas species of the gaseous sample 10.

Specifically, in this specific example, the filter elements 52 can include a notch filter element 54 to remove optical power within the excitation spectra of the first and second optical pulses. The filter elements 52 can also include a fluorescence and/or Raman pass filter 56 which lets pass optical power within spectral band(s) associated with predetermined fluorescence and/or Raman scattering bands of the given gas species. In these embodiments, the lifetime of the fluorescence is generally considerably smaller than the time delay between the first and second optical pulses.

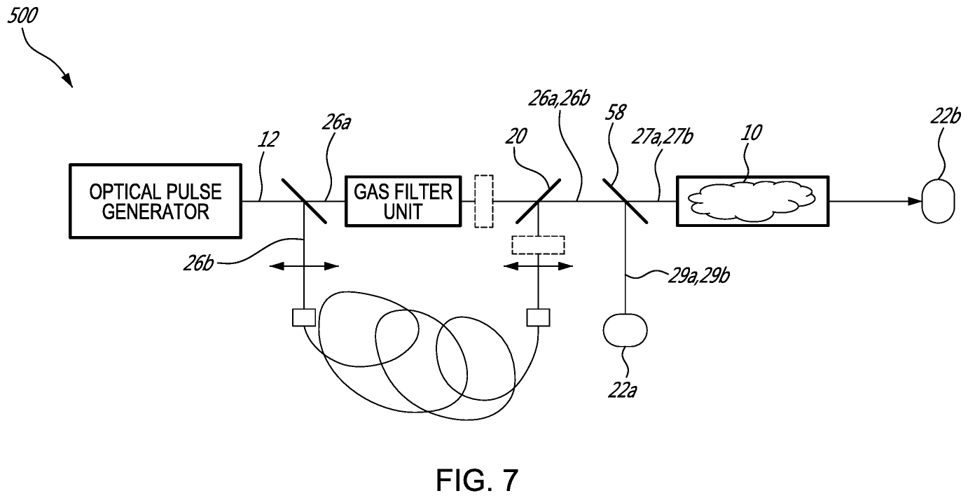

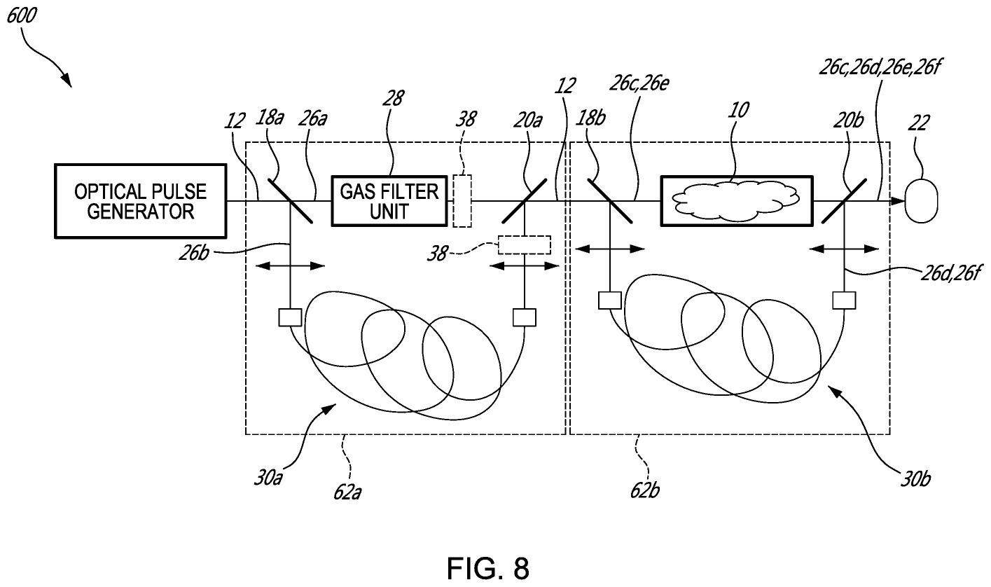

FIG. 7 shows another example of a system 500 for detecting a given gas species present in a gaseous sample 10. As depicted in this embodiment, the gaseous sample 10 is downstream from the optical beam combiner 20.

The system 500 of FIG. 7 is similar to the system 100 of FIG. 1. However, in this specific example, the system 500 has more than one optical detector 22. More specifically, the system 500 has a reference optical detector 22a and a measurement optical detector 22b.

As shown, the system 500 has an additional optical beam splitter 58 disposed along the first and second arm paths 26a and 26b, and between the optical beam combiner 20 and the gaseous sample 10. The additional optical beam splitter 58 is configured for splitting the first and second arm paths 26a, 26b into first and second measurement paths 27a, 27b, respectively, leading to the measurement optical detector 22b and into first and second reference paths 29a, 29b, respectively, leading to the reference optical detector 22a. As shown, the gaseous sample 10 is across the first and second measurement paths 27a and 27b.

As such, the first and second optical pulses are split into reference optical pulses directed along the first and second reference paths 29a and 29b, and measurement optical pulses directed along the first and second measurement paths 27a and 27b.

In this way, the computer (not shown) can be configured to detect the given gas species in the gaseous sample 10 based on signal values of the reference optical pulses as measured by the reference optical detector 22a and on signal values of the measurement optical pulses as measured by the measurement optical detector 22b.

FIG. 8 shows another example of a system 600 for detecting a given gas species present in a gaseous sample 10. In this embodiment, the optical beam splitter will now be referred to as a first optical beam splitter 18a, the time delay unit to as a first time delay unit 30a and the optical beam combiner to as a first optical beam combiner 20a.

As depicted, the first optical beam splitter 18a, the first time delay unit 30a, the optical gas filter unit 28 and the first optical beam combiner 20a collectively form a first optical assembly 62a which is upstream from the gaseous sample 10.

In this specific embodiment, the system 600 also has a second optical assembly 62b which is downstream from the first optical assembly 62a and upstream from the optical detector 22.

More specifically, the second optical assembly 62b has a second optical beam splitter 18b which is configured for splitting the first arm path 26a into third and fourth arm paths 26c and 26d, and for splitting the second arm path 26b into fifth and sixth arm paths 26e and 26f.

As shown, the third and fifth arm paths 26c and 26e have the gaseous sample 10 while the fourth and sixth arm paths 26d and 26f have a second time delay unit 30b. The second time delay unit 30b can also be called the reference unit. As discussed above, the second time delay unit 30b can also be disposed along the third and fifth arm paths 26c and 26e in other embodiments. The second optical assembly 62b also has a second optical beam combiner 20b configured for combining the third, fourth, fifth and sixth arm paths 26c, 26d, 26e and 26f towards the optical detector 22.

With such a configuration, once redirected by the first optical beam combiner 20a, the first and second optical pulses are each split into two optical pulses by the second optical beam splitter 18b. In this way, the first optical pulse is split into a third optical pulse directed along the third arm path 26c and a fourth optical pulse directed along the fourth arm path 26d. Similarly, the second optical pulse is split into a fifth optical pulse directed along the fifth arm path 26e and a sixth optical pulse directed along the sixth arm path 26f. As can be understood, both the third and the fifth optical pulses are propagated through the gaseous sample 10 while both the fourth and sixth optical pulses are propagated along the second time delay unit 30b.

When the time delays caused by both the first and second time delay units 30a and 30b are satisfactorily selected, the third, fourth, fifth and sixth optical pulses reach the optical detector 22 at different moments to provide signal values associated to each one of these four optical pulses. Depending on the time delay Δτ caused by the first time delay unit 30a and on the time delay Δτ′ caused by the second time delay unit 30b, the order in which these four optical pulses reach the optical detector 22 can vary.

For instance, in a specific embodiment, the time delay Δτ′ of the second time delay unit 30b is at least twice as long as the time delay Δτ of the first time delay unit 30a. In this way, the third and fifth optical pulses reach the optical detector 22 first and then the fourth and sixth optical pulses reach the optical detector 22, allowing the optical detector 22 to measure four temporally-spaced signal values. However, in other embodiments, it is noted that the time delay Δτ of the first time delay unit 30a can be at least twice as long as the time delay Δτ′ of the second time delay unit 30b.

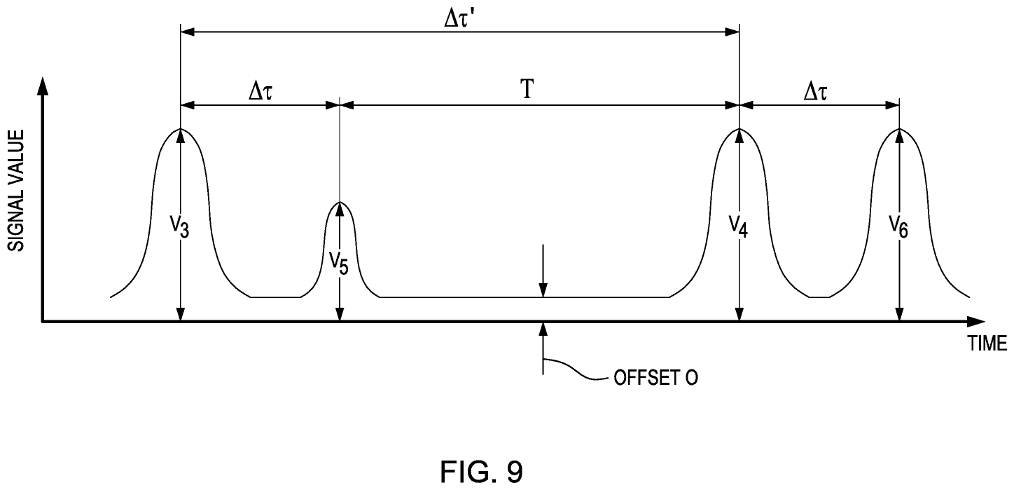

FIG. 9 is a graph showing signal as function of time as the four optical pulses described with reference to FIG. 8 reach the optical detector 22.

In this specific embodiment, the time delay Δτ′ of the second time delay unit 30b exceeds the time delay Δτ of the first delay unit by a given time period T. In this way, the third optical pulse reaches the optical detector 22 first, the fifth optical pulse reaches the optical detector 22 second, the fourth optical pulse reaches the optical detector 22 third and the sixth optical pulse reaches the optical detector 22 last. As shown, the third and fifth optical pulses are temporally spaced apart by the time delay Δτ. Similarly, the fourth and sixth optical pulses are temporally spaced apart by the time delay Δτ. The third and fourth are temporally spaced apart by the time delay Δτ ′. Similarly, the fifth and sixth optical pulses are temporally spaced apart by the time delay Δτ ′. As shown, the fourth and fifth optical pulses are temporally spaced apart by the given time period T, and allows to determine an offset value O at the optical detector 22, which can then be used in the measurement of the concentration of the given gas species.

In this embodiment, the third and fifth optical pulses can be referred to as a pair of measurement optical pulses, as they both propagate through the gaseous sample 10. In contrast, the fourth and sixth optical pulses can be referred to as a pair of reference optical pulses as they both propagate along the second time delay unit 30b, thus bypassing the gaseous sample 10.

In this case, the optional, electronically controllable, optical attenuator(s) 38 have been configured to equalize the energies of the two optical pulses going through the reference unit 30b so their signal values be similar to one another in order to null the system 600. Preferably, the difference in the fourth and sixth signal values is used, in real time, to adjust the optical attenuator(s) 38 in order to null the system 600.

More specifically, FIG. 9 shows signal as function of time as the third and fifth optical pulses reach the optical detector 22 when the gaseous sample 10 does contain the given gas species. Accordingly, the presence of the given gas species can be detected as the signal value V₅ of the fifth optical pulse will tend to decrease as function of the concentration of the given gas species, thus decreasing relatively to the signal value V₃ of the third optical pulse. The presence of the reference pulses can allow the system 600 to correct for variations in the nulling of the system 600, either through control of attenuator(s) 38 or through computation. For instance, the ratio of the difference of the signal values V₄ and V₆ of the fourth and sixth optical pulses over their sum (with these last two corrected for the offset O), can be used to correct the measured relative difference in the signal values V₃ and V₅ of the third and fifth optical pulses.

In some embodiments, the time delays Δτ and/or Δτ′ are selected so that the optical pulses in a pair having the lesser signal reach the optical detector 22 first, be it the pair of optical reference pulses or the pair of measurement optical pulses.

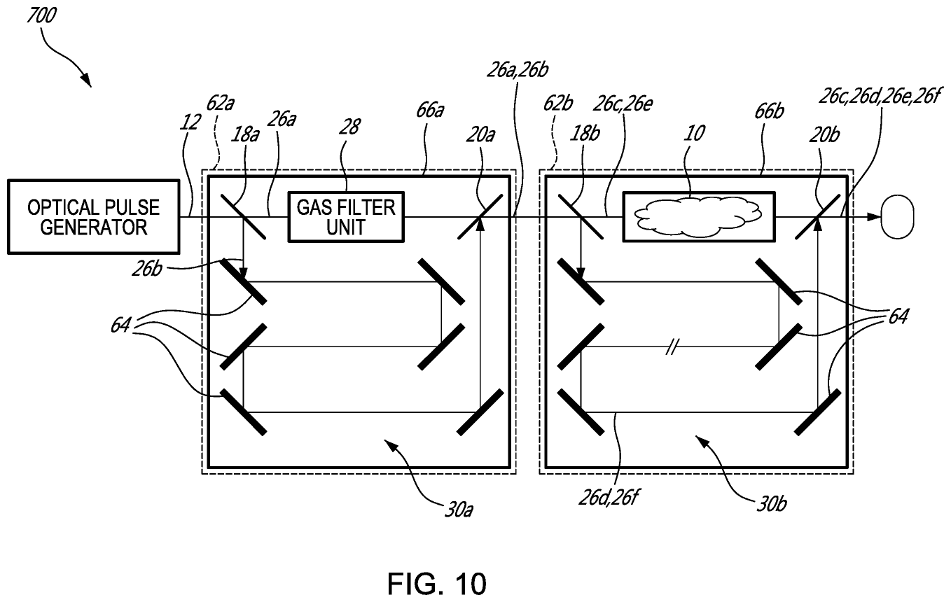

FIG. 10 shows another example of a system 700 for detecting a given gas species present in a gaseous sample 10. As depicted in this embodiment, the gaseous sample 10 is downstream from the first optical assembly 62a.

The system 700 of FIG. 10 is similar to the system 600 of FIG. 8. However, in this specific example, the first and second time delay units 30a and 30b are different. As shown, instead of using lengths of optical fiber to cause the time delays Δτ and Δτ′, these time delays are obtained by free-space propagations along the second, fourth and sixth arm paths 26b, 26d and 26f. More specifically, reflective surfaces 64 such as mirrors are provided in each one of the second, fourth and sixth arm paths 26b, 26d and 26f to make the free-space paths more compact. The reflective surfaces 64 of the second, fourth and sixth arm paths 26b, 26d and 26f can each be in the form of a multi-reflection cell, when the multiple reflections are on the same two reflective surfaces (e.g., two concave reflective surfaces facing each other).

Moreover, in this embodiment, a first housing 66a encloses the first optical assembly 62a while a second housing 66b encloses the second optical assembly 62b. In this embodiment, both the first and second delay units 30a and 30b are preferably under vacuum.

As discussed above, although the first optical assembly 62a is upstream from the second optical assembly 62b in both the system 600 of FIG. 8 and the system 700 of FIG. 10, the first optical assembly 62a can be downstream from the second optical assembly 62b in alternate embodiments.

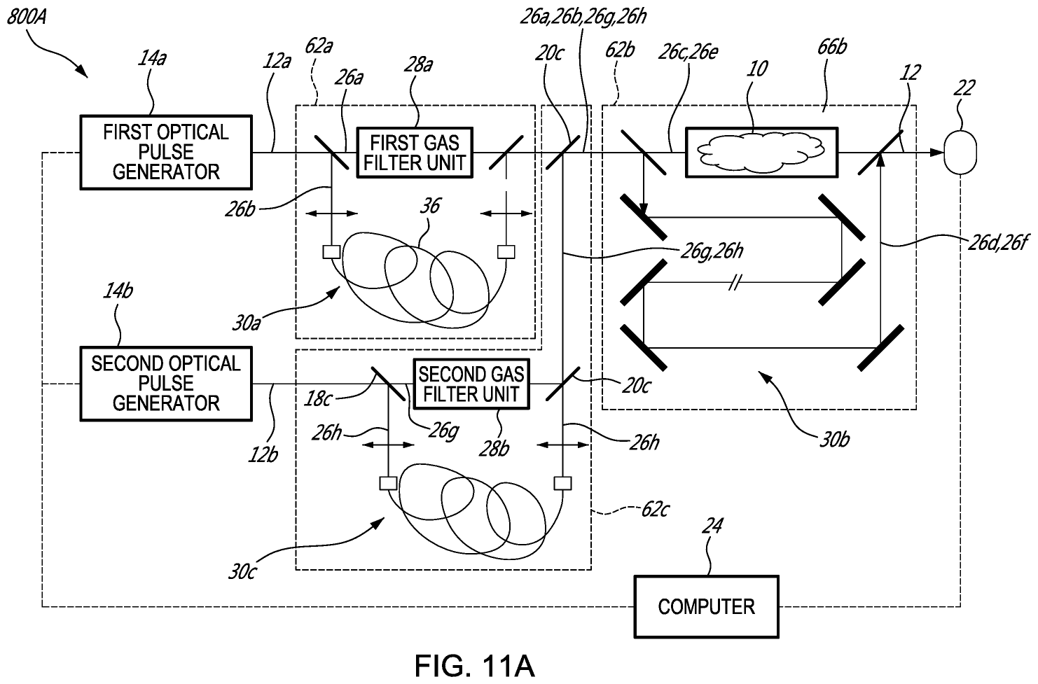

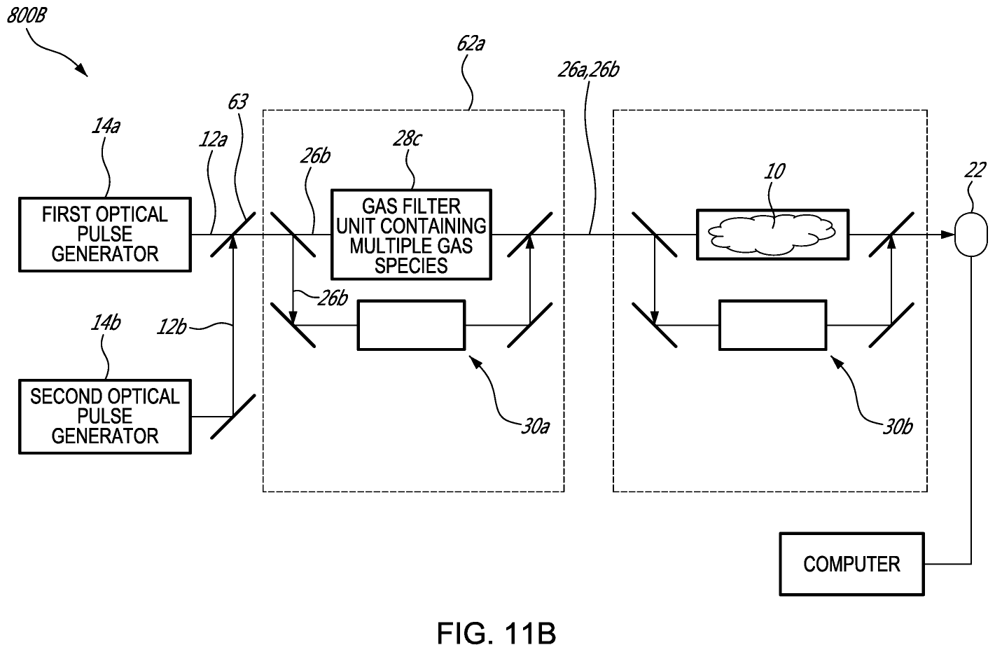

FIG. 11A shows another example of a system 800A for detecting first and second gas species present in a gaseous sample 10. As depicted in this embodiment, the gaseous sample 10 is downstream from the first and second arm paths 26a and 26b.

The system 800A of FIG. 11A is similar to the system 600 of FIG. 8. However, in this specific example, the first time delay unit 30a comprises a length of optical fiber 36 whereas the second time delay unit 30b comprises a multi-reflection cell, as they both achieve a similar function.

In addition, in this specific embodiment, a third optical assembly 62c is provided. More specifically, the third optical assembly 62c is disposed in parallel with the first optical assembly 62a. The third optical assembly 62c has a third optical beam splitter 18c which is configured for splitting a second primary optical path 12b into seventh and eighth arm paths 26g and 26h. The seventh arm path 26g has a second optical gas filter unit 28b containing the second gas species. The second gas species has a spectral absorption band within at least some of the excitation spectrum of a primary optical pulse which may differ from the spectral absorption band of the first gas species contained in the first optical gas filter unit 28a. As shown, the eighth arm path 26h has a third time delay unit 30c. The third optical assembly 62c also has third optical beam combiners 20c which are configured for combining the seventh and eighth arm paths 26g and 26h to one another towards the second optical assembly 62b.

In this embodiment, the computer 24 is configured for selectively initiating the propagation of a primary optical pulse along either the first primary optical path 12a or the second primary optical path 12b at any given time. Moreover, the computer 24 is configured for detecting one of the first gas species and the second gas species in the gaseous sample 10 based on the signal values measured by the optical detector 22. Indeed, when a primary optical pulse is propagated towards the first primary optical path 12a, the presence and/or concentration of the first gas species in the gaseous sample 10 can be detected. Likewise, when a primary optical pulse is propagated towards the second primary optical path 12b, the presence and/or concentration of the second gas species in the gaseous sample 10 can be detected.

In this embodiment, the optical pulse generator is a first optical pulse generator 14a and the system 800A has a second optical pulse generator 14b. In this case, the computer 24 can selectively control which one of the first and second optical pulse generators 14a and 14b generate the primary optical pulse, and in turn, control which one of the first and second gas species can be detected.

In other embodiments, only one optical pulse generator can be used to detect the presence of the first and second gas species in the gaseous sample 10. More specifically, the single optical pulse generator can be configured for selectively propagating the primary optical pulse along either one of the first and third optical assemblies via an optical switch (not shown). In these specific examples, the computer 24 can selectively control the optical switch so as to propagate the primary optical pulse towards a selected one of the first and third optical assemblies 62a and 62c. As can be understood, the optical switch can be an electro-optical switch with no moving parts.

FIG. 11B is a schematic view of another example of a system 800B for detecting first and second gas species present in a gaseous sample 10. As shown in this example, the system 800B has first and second optical pulse generators 14a and 14b which are configured to generate primary optical pulses having different spectral contents and propagating along a respective one of the first and second primary optical paths 12a and 12b. In this example, the first and second primary optical paths 12a and 12b are combined to one another via a dichroic beam combiner 63 upstream from the optical unit 62a. In contrast with the embodiment of FIG. 11A, the first and second gas species to be measured are both contained inside a single filter gas unit 28c attenuating the primary optical pulse at the absorption spectral bands of the first and second gas species.

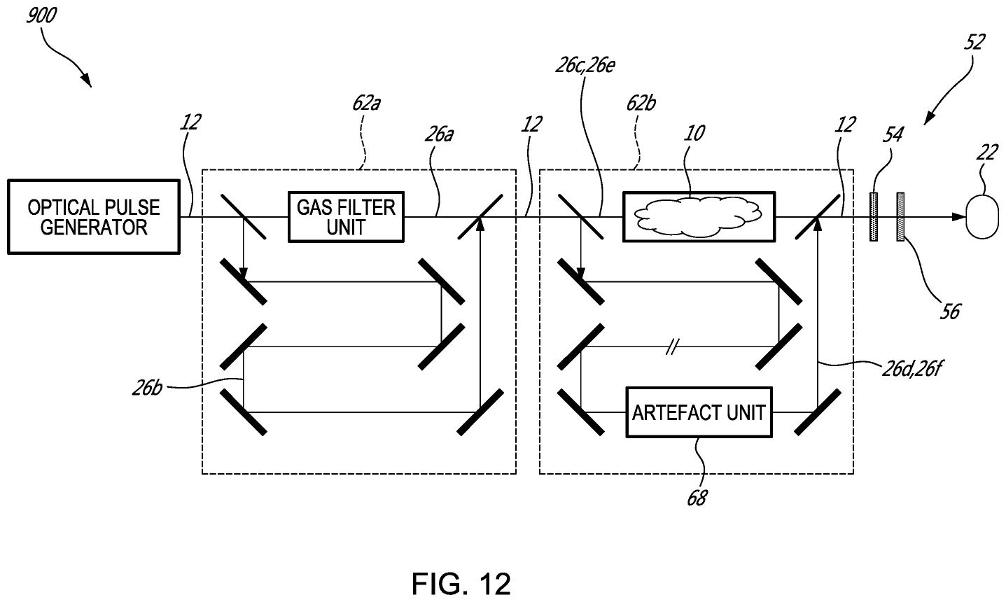

FIG. 12 shows another example of a system 900 for detecting a given gas species present in a gaseous sample 10. As depicted in this embodiment, the gaseous sample 10 is downstream from the first and second arm paths 26a and 26b.

The system 900 of FIG. 12 is similar to the system 700 of FIG. 10. However, in this specific example, the fourth arm path 26d and the sixth arm path 26f of the second optical assembly 62b have an artefact unit 68 so as to produce known and calibrated fluorescence and/or Raman scattering as the optical pulses propagating along the fourth and sixth arm paths 26d and 26f interact with the given gas species of the artefact unit 68. For example, if the given gas species is benzene, then the artefact unit 68 would have a known amount of benzene. In this case, the benzene would be in clean air with the same composition, pressure and temperature to that of the gaseous sample 10, except for minor pollutants.

As shown, and similarly to the system 400 of FIG. 6, the system 900 has filter elements 52 which are configured to filter out optical power within the excitation spectrum of the four excitation optical pulses. In this way, the optical detector 22 can measure signals which are indicative of fluorescence and/or Raman scattering produced by any one of the third, fourth, fifth and sixth optical pulses reaching the optical detector 22.

The filter elements 52 can include a notch filter element 54 to remove optical power within the excitation spectrum of the third, fourth, fifth and sixth optical pulses. The filter elements 52 can also include a fluorescence and/or Raman pass filter 56 which lets pass optical power within spectral band(s) associated with fluorescence and/or Raman scattering of the given gas species. Moreover, in this specific embodiment, the optical detector 22 is configured for measuring signal values in the spectral bands which encompass the fluorescence and/or the Raman scattering which are expected from the given gas species. Moreover, in these embodiments, the lifetime of the fluorescence should be considerably shorter than the time delay between the first and second optical pulses.

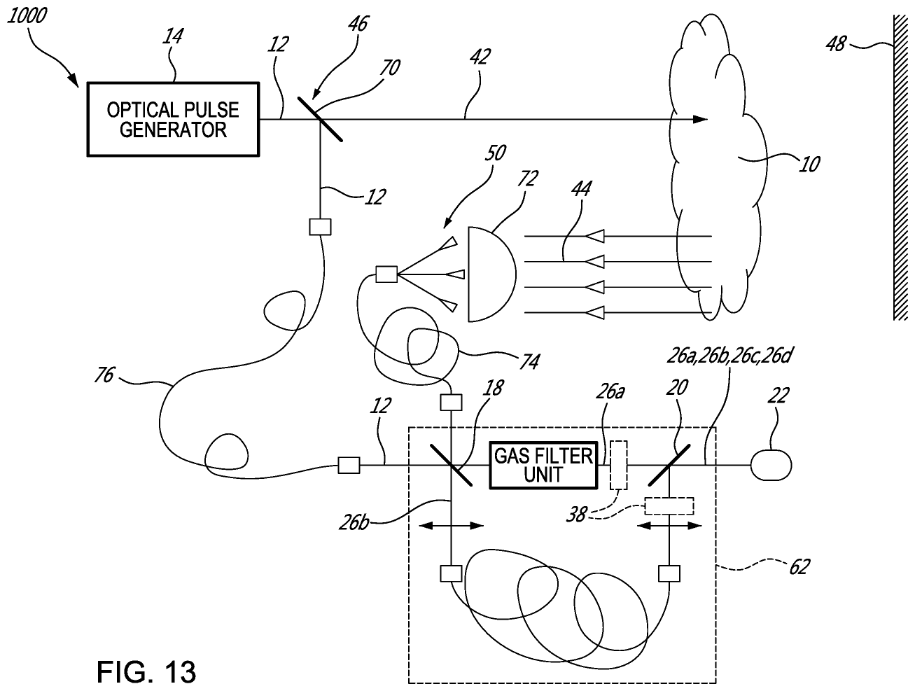

FIG. 13 shows another example of a system 1000 for detecting a given gas species present in a gaseous sample 10.

The system 1000 of FIG. 13 is similar to the system 200 of FIG. 4 in that the gaseous sample 10 is upstream from the first and second arm paths 26a and 26b of the optical assembly 62. However, in this specific example, the system 1000 has an emission assembly 46 configured to route the optical pulse(s) along an outgoing path 42 and towards a distant target 48 and a collection assembly 50 configured to route the returning optical pulse(s) from a return path 44 towards the optical detector 22. As shown, the gaseous sample 10 is distributed along the outgoing path 42 and the return path 44 in this example. However, it will be understood that the gaseous sample 10 can be disposed along a portion of the outgoing path 42 and along a portion of the return path 44 in some other embodiments. As shown, the returning optical pulses thereby follow third and fourth arm paths 26c and 26d which lead to the optical detector 22.

More specifically, the emission assembly 46 has an emission optical beam splitter 70 which is upstream from the optical beam splitter 18. The emission optical beam splitter 70 can be provided in the form of a 95/5 optical beam splitter such that the primary optical pulse generated by the optical pulse generator 14, and propagated along the primary optical path 12, is split into a primary measurement optical pulse propagating along the outgoing path 42 and a primary reference optical pulse coupled to emission optical fiber 76. In this example, the primary measurement optical pulse carries more optical power than the primary reference optical pulse, which is then a relatively small portion of the primary optical pulse.

As shown, the primary measurement optical pulse is directed towards the distant target 48, and returns as a reflection or as scattering towards the collection assembly 50. In this embodiment, the collection assembly 50 has a light converging element 72 configured to receive the primary measurement optical pulse and to couple it to the collection optical fiber 74 which leads to the optical beam splitter 18 of the optical assembly 62. In this way, the optical beam splitter 18 splits the primary measurement optical pulse into a first measurement optical pulse directed along the first arm path 26a and a second measurement optical pulse along the second arm path 26b. Both the first and second measurement optical pulses are routed towards the optical detector by the optical beam combiner 20.

The primary reference optical pulse propagates in the emission optical fiber 76 which is optically coupled to the optical beam splitter 18 of the optical assembly 62. Accordingly, the primary reference optical pulse is split into a first reference optical pulse directed along the first arm path 26a and a second reference optical pulse directed along the second arm path 26b. Both the first and second optical pulses are routed towards the optical detector 22 by the optical beam combiner 20.

As can be understood, similarly to other embodiments described herein, the optical detector 22 is configured to sequentially measure the signal values of the first and second measurement optical pulses and the signal values of the first and second reference optical pulses. The signal values of the first and second measurement optical pulses will depend on the presence and/or concentration of the given gas species along the outgoing and return paths 42 and 44.

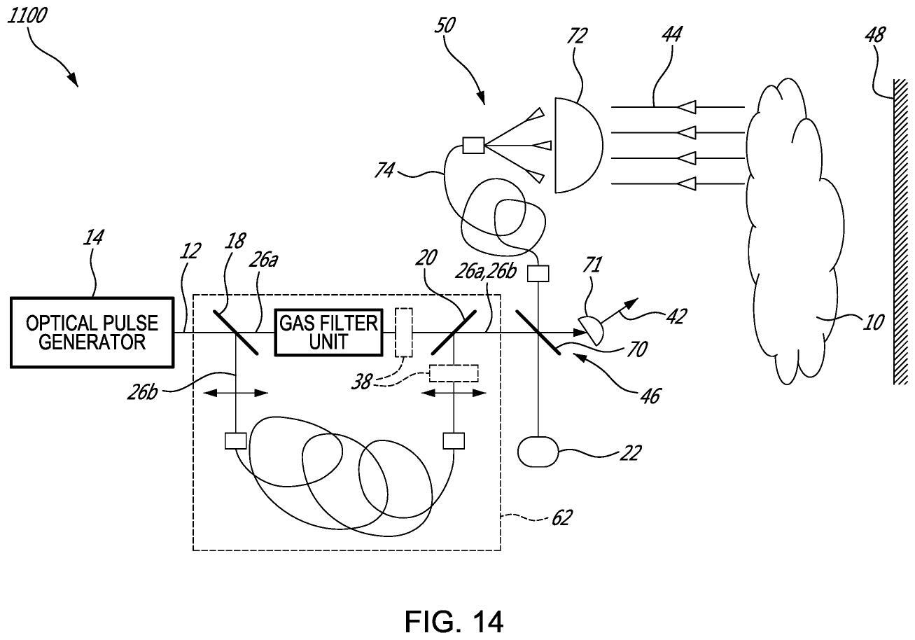

FIG. 14 shows another example of a system 1100 for detecting a given gas species present in a gaseous sample 10.

The system 1100 of FIG. 14 is similar to the system 100 of FIG. 1 in that the gaseous sample 10 is downstream from the optical beam combiner 20 of the optical assembly 62. However, in this specific example, the system 1100 has an emission assembly 46 configured to redirect first and second arm paths 26a and 26b along the outgoing path 42 directed towards the distant target 48 and a collection assembly 50 configured to redirect the return path 44 incoming from the distant target 48 towards the optical detector 22. A redirecting element 71 may be used to direct the outgoing path 42 towards the given target 48, wherever it may be. As shown, the gaseous sample 10 is distributed along the outgoing and return paths 42 and 44 in this example.

As discussed above, in this configuration, the primary optical pulse generated by the optical pulse generator 14 and propagated along the primary optical path 12 is split into a first optical pulse along the first arm path 26a and a second optical pulse along the second arm path 26b by the optical beam splitter 18. Once combined to one another by the optical beam combiner 20, an emission optical beam splitter 70 of the emission assembly 46, downstream from the optical beam combiner 20, splits the first optical pulse into a first measurement optical pulse along the outgoing path 42 and a first reference optical pulse routed to the optical detector 22. Similarly, the optical beam splitter 70 splits the second optical pulse into a second measurement optical pulse along the outgoing path 42 and a second reference optical pulse towards the optical detector 22. It is noted that the optical beam splitter 70 is optional and can be omitted in at least some embodiments.

Again in this example, the optical beam splitter 70 can be provided in the form of a 95/5 optical beam splitter where only 5% of the optical power is directed towards the optical detector 22.

As shown, the first and second measurement optical pulses are directed towards the distant target 48, and return as a reflection or as scattering towards the collection assembly 50. In this embodiment, the collection assembly 50 has a light converging element 72 configured to receive the first and second measurement optical pulses and to couple them to the collection optical fiber 74 which leads to the emission optical beam splitter 70. As such, in this specific embodiment, both the first and second measurement optical pulses pass through the optical beam splitter 70 prior to reaching the optical detector 22.

As can be understood, similarly to other embodiments described herein, the optical detector 22 is configured to measure the signal values of the four temporally delayed optical pulses. In this embodiment, the signal values of the first and second measurement optical pulses will depend on the presence and/or concentration of the given gas species along the outgoing and return paths 42 and 44.

As will be understood, as shown in FIGS. 13 and 14, the systems 1000 and 1100 are configured such that the optical detector 22 measures signal values of reference optical pulses, which do not pass through the gaseous sample 10. Moreover, in embodiments where the systems 1000 and 1100 were to be nulled, the signal values of the reference optical pulses can be adjusted via the attenuators 38 disposed in either one of both of the first and second arm paths 26a and 26b.

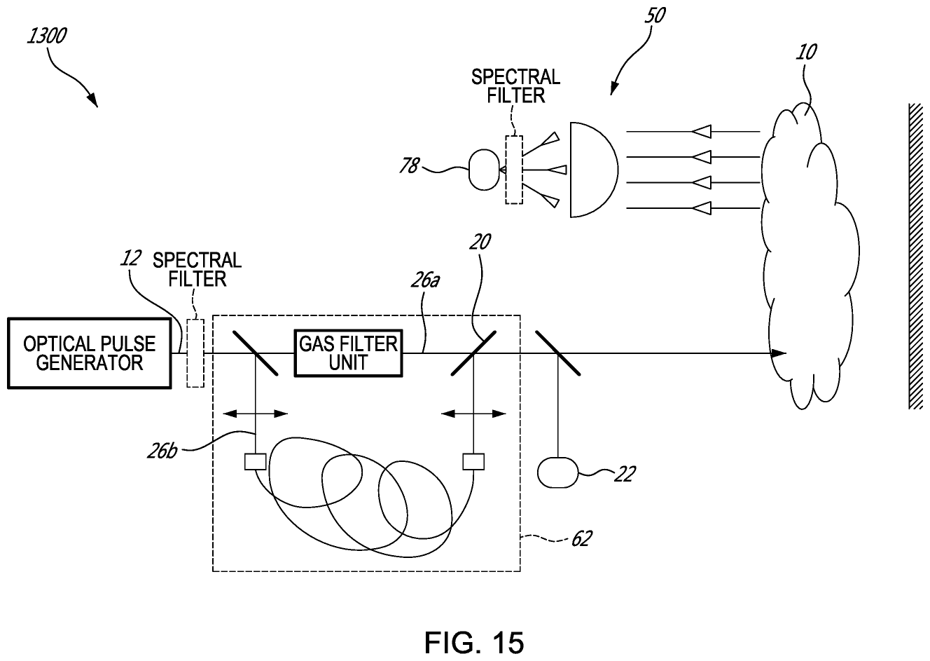

FIG. 15 shows another example of a system 1300 for detecting a given gas species present in a gaseous sample 10.

The system 1300 of FIG. 15 is similar to the system 1100 of FIG. 14 in that the gaseous sample 10 is downstream from the optical beam combiner 20 of the optical assembly 62. However, in this specific example, the collection assembly 50 is optically coupled to a camera 78 instead of leading to the optical detector 22. The system 1300 can thus be used in an imaging mode. Accordingly, the camera 78 can generate an image in which pixels are indicative of the presence and/or concentration of the given gas species at specific spaced-apart locations. For instance, in some embodiments, the camera 78 is a gated imaging detector used in the open path scenario. In some embodiments, the gated imaging detector can have at least two side-by-side integration gates, such as an interline CCD or the double gate sensor (epc502) from ESPROS (Switzerland). In these embodiments, the first gate images with the first measurement optical pulse that went through the first arm 26a, while the second gate images with the second measurement optical pulse that went through the second arm 26b, or vice versa. In such embodiments, the optical detector 22 is only used to detect the first and second reference pulses.

In view of the aforementioned, the systems presented above can perform a method of detecting a given gas species present in a gaseous sample. More specifically, this method has a step of splitting a primary optical pulse into a first optical pulse and a second optical pulse, the primary optical pulse having a duration and carrying optical power within an excitation spectrum encompassing at least one absorption spectral band of the given gas species, the first optical pulse being propagated across an optical gas filter unit containing an amount of the given gas species to be detected and attenuating the first optical pulse at the at least one absorption band, one of i) the primary optical pulse and ii) the first and second optical pulses being propagated across the gaseous sample, and temporally delaying the first and second optical pulses from one another; a step of measuring measurement signal values of the temporally delayed first and second optical pulses using a measurement optical detector; and a step of detecting the presence of the given gas species in the gaseous sample based on the measurement signal values. Other optional steps of this method are apparent from the description of the systems described herein.

As can be understood, the various embodiments described above and illustrated are intended to be exemplary only. For instance, the system can be used to detect a given gas species generated by spills or leaks that can occur in infrastructures such as pipelines, pumping stations, refineries, extraction sites and storage sites. As can be understood from above, the detection of spills or leaks can be made remotely, without disrupting the normal operation of the infrastructure, and at any desired frequency (e.g., once per day, once per month, once per year). Indeed, in some specific embodiments, the system is mounted to an aircraft which can be used to fly over the given infrastructure. The aircraft is a manned aircraft such as an airplane or a helicopter in some embodiments whereas the aircraft is an unmanned aircraft such as a drone in some other embodiments. Any other suitable type of aircraft can be used. As will be understood, the term “spill” is used herein so as to encompass any kind of spill such as pools, slicks and gas/vapor leaks. The spill can be caused by a leak in a pipeline, buried or above ground, in a storage facility (tank, tank farm, truck, ship and the like), by spilling a product in transfer operations, or from vapors originating from open liquid/solid containers or leaks in vapor phase products in storage containers.

The primary optical pulse can have any suitable temporal or spectral shape. For instance, in some embodiments the primary optical pulse has a Gaussian profile while in some other embodiments it has a square profile. Depending on the embodiment, an optical filter can be added after the optical pulse generator, before the gaseous sample, after the gas cell or before the optical detector, for filtering out optical power outside the excitation spectrum of the primary optical pulse. The scope is indicated by the appended claims.

What is claimed is:

1. A method for detecting a given gas species present in a gaseous sample, the method comprising:
splitting a primary optical pulse into a first optical pulse and a second optical pulse, the primary optical pulse having a duration and carrying optical power within an excitation spectrum encompassing at least one absorption spectral band of the given gas species, the first optical pulse being propagated across an optical gas filter unit containing an amount of the given gas species to be detected and attenuating the first optical pulse at the at least one absorption band, one of i) the primary optical pulse and ii) the first and second optical pulses being propagated across the gaseous sample, and temporally delaying the first and second optical pulses from one another;
measuring measurement signal values of the temporally delayed first and second optical pulses using a measurement optical detector; and
detecting the presence of the given gas species in the gaseous sample based on the measurement signal values.

2. The method of claim 1 further comprising measuring an offset value of the measurement optical detector, the offset value being indicative of the value measured by the measurement optical detector in absence of any optical pulse, said detecting comprising determining a concentration value of the given gas species present in the gaseous sample based on the measurement signal values and on the offset value of the measurement optical detector.

3. The method of claim 1 or 2 further comprising measuring reference signal values of the temporally delayed first and second optical pulses using a reference optical detector, the reference signal values being indicative of the values of the first and second optical pulses in absence of any propagation across the gaseous sample, said detecting being further based on the reference signal values.

4. The method of claim 3 wherein the measurement signal values and the reference signal values are indicative of one of a peak optical power and energy of the corresponding optical pulses.

5. The method of claim 1 wherein the given gas species is distributed along one of an open path and a gas cell.

6. The method of claim 1 further comprising generating the primary optical pulse, said generating and said measuring being synchronized with one another.

7. The method of claim 1 or 2 wherein as the first and second optical pulses are propagated across the gaseous sample at least one of fluorescence and Raman scattering is produced, the method further comprising, subsequent to said production and prior to said measuring, filtering out optical power within said excitation spectrum, the measured signal values being indicative of said at least one of fluorescence and Raman scattering.

8. The method of claim 1 wherein the temporal delay exceeds the duration of the primary optical pulse.

9. A system for detecting a given gas species present in a gaseous sample, the system comprising:
an optical pulse generator configured to generate a primary optical pulse directed along a primary optical path, the primary optical pulse having a duration and carrying optical power within an excitation spectrum encompassing at least one absorption spectral band of the given gas species;
an optical beam splitter configured for splitting the primary optical pulse into a first optical pulse directed along a first arm path and a second optical pulse directed along a second arm path, the first arm path having an optical gas filter unit containing an amount of the given gas species to be detected and configured for attenuating the first optical pulse at the at least one absorption band, one of the first and second arm paths having a time delay unit being configured to cause a time delay between the first and second optical pulses, one of i) the primary optical path and ii) the first and second arm paths extending across the gaseous sample;
a measurement optical detector configured for measuring measurement signal values of the first and second optical pulses propagating along the first and second arm paths; and
a computer detecting the given gas species in the gaseous sample based on the measurement signal values.

10. The system of claim 9 wherein the measurement optical detector measures an offset value in absence of any optical pulse, said computer determining a concentration value of the given gas species in the gaseous sample based on the measurement signal values and on the offset value of the measurement optical detector.

11. The system of claim 9 or 10 further comprising a reference optical detector measuring reference signal values of the temporally delayed first and second optical pulses, the reference signal values being indicative of the values of the first and second optical pulses in absence of any propagation across the gaseous sample, said detecting being further based on the reference signal values.

12. The system of claim 11 wherein the measurement and reference optical detectors are a same optical detector.

13. The system of claim 9 wherein the gaseous sample is at least one of enclosed in a transparent chamber and distributed along an open path.

14. The system of claim 9 further comprising an emission assembly configured to redirect one of i) the primary optical path and ii) the first and second arm paths along an outgoing open path directed towards a distant target and a collection assembly configured to redirect a return path from the distant target towards the measurement optical detector, the gaseous sample being distributed along at least one of the outgoing open path and the return path.

15. The system of claim 9 further comprising an optical beam combiner downstream from said optical gas filter unit and said time delay unit and being configured for combining the first and second arm paths along a common path.

16. The system of claim 15 wherein the gaseous sample is downstream from the optical beam combiner and across the first and second arm paths.

17. The system of claim 14 wherein at least one of fluorescence and Raman scattering is produced as the first and second optical pulses propagate across the gaseous sample, the system further comprising at least one filter element downstream from the gaseous sample, upstream from the measurement optical detector and along the first and second arm paths, the at least one filter element being configured to filter out optical power within said excitation spectrum, the measured signal values being indicative of the at least one of fluorescence and Raman scattering.

18. The system of claim 15 wherein the optical beam splitter is a first optical beam splitter, the time delay unit is a first time delay unit and the optical beam combiner is a first optical beam combiner, the first optical beam splitter, the first time delay unit and the first optical beam combiner collectively forming a first optical assembly, the system further comprising a second optical assembly having a second optical beam splitter being configured for splitting the first arm path in third and fourth arm paths and for splitting the second arm path in fifth and sixth arm paths, the gaseous sample being across the third and fifth arm paths, one of a) the third and fifth arm paths and b) the fourth and sixth arm paths having a second time delay unit.

19. The system of claim 18 wherein the third, fourth, fifth and sixth arm paths lead to the measurement optical detector.

20. The system of claim 18 wherein the second time delay unit is configured to cause a time delay being at least twice as long as the time delay caused by the first time delay unit.
